(19) United States
(12) Patent Application Publication
(10) Pub. No.: US 2012/0178108 A1
(43) Pub. Date: Jul. 12, 2012

(54) IMMUNODETECTABILITY

(75) Inventors: Mathias Uhlén, Stocksund (SE); Jochen Schwenk, Stockholm (SE)

(73) Assignee: ATLAS ANTIBODIES AB, Stockholm (SE)

(21) Appl. No.: 13/379,798

(22) PCT Filed: Jun. 26, 2009
(86) PCT No.: PCT/SE2009/000328

§ 371 Date: Jan. 23, 2012

(51) Int. Cl. G01N 21/64; G01N 33/82; G01N 33/53

(52) U.S. Cl. 435/7.92; 436/501

(57) ABSTRACT

In the present disclosure, there is provided a method for improving the immunodetectability of at least one protein in an optionally diluted sample of blood, serum or plasma, comprising a step of heating the sample to a temperature of 64-85° C. prior to a contact between the sample and at least one affinity ligand for detection and/or quantification of the at least one protein.

### FIELD OF THE INVENTION

The present invention relates to the field of detection of proteins in blood or blood-derived samples.

### BACKGROUND

Currently, a widely used and well-accepted technology in proteome analysis is mass spectrometry, often employed in combination with 2D-gel electrophoresis or chromatography techniques. However, recent developments of miniaturized and parallelized technology platforms have opened the possibility to support and supplement mass spectrometric analysis.

### SUMMARY

Firstly, there is provided a method for improving the immunodetectability of at least one protein in an optionally diluted sample of blood, serum or plasma, comprising a step of heating the sample to a temperature of 50-85° C. prior to a contact between the sample and at least one affinity ligand.

Secondly, there is provided a method for detecting and/or quantifying proteins in blood, serum or plasma from a subject:
a) providing a first and a second optionally diluted sample of blood, serum or plasma from the subject;
b) heating the first sample to a temperature of 50-85° C.;
c) contacting the first sample, subsequent to the heating of b), with at least one affinity ligand capable of selective interaction with a target protein which immunodetectability is increased by the heating;
d) contacting the second sample, which has not been subjected to the heating, with at least one affinity ligand capable of selective interaction with a target protein which immunodetectability is not increased by the heating;
e) detecting interactions between the antibodies and the corresponding target proteins formed in step c) and d), thereby detecting and/or quantifying proteins in the blood, serum or plasma.

Thirdly, there is provided a method for identification of a biomarker of a medical condition, comprising
a) providing blood, serum or plasma samples from a first group of subjects having the medical condition and a second group of subjects not having the medical condition,
b) heating the samples, optionally after dilution, to a temperature of 50-85° C.,
c) contacting the samples with at least one affinity ligand capable of selective interaction with at least one protein to determine the levels of the at least one protein in the respective groups,
d) comparing the levels to identify a protein which is occurs to a higher or lower degree in the sample from the first group than in the samples from the second group, thus identifying the biomarker of the medical condition.

Various embodiments of the above methods are described in more detail below.

### BRIEF DESCRIPTION OF THE FIGURES

In FIGS. 1-4, the solid line represents proteins in plasma and the dashed line represents proteins in serum.

FIG. 1 shows the number of proteins in plasma or serum exhibiting an at least two-fold increased immunodetectability (y-axis) after heat treatment at different temperatures (x-axis).

FIG. 2 shows the number of proteins in plasma or serum exhibiting an at least two-fold decreased immunodetectability (y-axis) after heat treatment at different temperatures (x-axis).

FIG. 3 shows the number of proteins in plasma or serum exhibiting an at least two-fold increased immunodetectability (y-axis) after heat treatment at different temperatures (x-axis).

FIG. 4 shows the number of proteins in plasma or serum exhibiting an at least two-fold decreased immunodetectability (y-axis) after heat treatment at different temperatures (x-axis).

FIG. 5 shows the signals from 6 different affinity ligands interacting with proteins in serum after treatment at 23° C., 56° C. and 72° C. The box plots are made from raw fluorescent signal intensity data and based on the result of three replicated analyses. MFI=median fluorescent intensity, AU=arbitrary unit.

FIG. 6 shows the signals from 6 different affinity ligands interacting with proteins in plasma after treatment at 23° C., 56° C. and 72° C. The box plots are made from raw fluorescent signal intensity data and based on the result of three replicated analyses. MFI=median fluorescent intensity, AU=arbitrary unit.

FIG. 7 shows the differences in levels of proteins in plasma from PSA low or high patients (PSA>60 ng/ml) after treatment at 72° C. Results were presented as a volcano plot, where the relative fold-change (x-axis) is plotted against significance (P-value) for t-test results (y-axis). A solid line represents a p-value <0.01, and a dashed line represents a p-value <0.05. The dotted box represents . . . .

FIG. 8 shows the differences in levels of proteins in plasma from PSA low or high patients (PSA >60 ng/ml) after treatment at 23° C. Results were presented as a volcano plot, where the relative fold-change (x-axis) is plotted against significance (P-value) for t-test results (y-axis). A solid line represents a p-value <0.01, and a dashed line represents a p-value <0.05. The dotted box represents the magnification of the area of the volcano plot where protein with smaller fold-change and significance values are located.

### DETAILED DESCRIPTION

As a first aspect of the present disclosure, there is thus provided a method for improving the immunodetectability of at least one protein in an optionally diluted sample of blood, serum or plasma, comprising a step of heating the sample to a temperature of 50-85° C. prior to a contact between the sample and at least one affinity ligand.

As a configuration of the first aspect, there is provided a method for detecting protein in a sample of blood, serum or plasma, comprising:
a) heating the sample to a temperature of 50-85° C.;
b) contacting the sample with at least one affinity ligand capable of selective interaction with a known target protein;
c) detecting interaction(s) between the at least one affinity ligand and the corresponding target protein(s) from the sample thereby detecting protein in the sample.

The present disclosure is based on the finding that some proteins of plasma and serum samples are detected by antibodies to a higher degree if the plasma or serum samples are heated before the analysis. Without being bound by any specific scientific theory, the interaction between antibodies and their corresponding epitopes in the serum or plasma proteins appears to be facilitated by the heating.

The finding of the present disclosure may entail a number of benefits. Using the heating, it may be possible to detect proteins that were previously not detectable using immunological methods. Consequently, a more comprehensive picture of the protein contents of complex biological samples may be obtained. Also, the heating may enable the detection of proteins which are present in such complex biological samples at low levels. This may be of particular interest since many interesting biomarkers have been reported to be found at lower concentration ranges. The heating of the present disclosure may thus be a useful tool for increasing the sensitivity of proteomic studies.

In the context of the present disclosure, the “immunodetectability” of a protein refers to the extent of which a linear or conformational epitope of the protein may be detected by an affinity ligand, such as an antibody, capable of selective interaction with such epitope.

Further, in the context of the present disclosure, “improving” the immunodetectability of the protein refers to increasing the signal (or output) from the protein in an analysis based on epitope recognition as compared to the signal (or output) from the protein when no heating has been performed. Consequently, to determine whether the immunodetectability has been improved, the signal from the protein in question may be measured in two samples from a patient taken at the same time, wherein one of the samples has been heated before the measurement. The immunodetectability is improved if the signal from the protein in the heated sample is higher than the signal from the protein in the non-heated sample. To improve the accuracy of such comparison, more than one measurement on each sample and/or measurements on more than one sample of each category may be performed. In some embodiments, the immunodetectability may be considered improved if the signal, in absolute values, has increased 1.5-fold or 2-fold.

The “contact between the sample and at least one affinity ligand” makes a detection and/or quantification of the protein(s) in the sample possible. Consequently, the selectivity of the affinity ligand may be employed for determining the presence and/or abundance of the protein which the affinity ligand recognizes. The contact may be performed using various set-ups and formats, as discussed further below.

As a second aspect of the present disclosure, there is provided a method for detecting and/or quantifying proteins in blood, serum or plasma from a subject:
a) providing a first and a second optionally diluted sample of blood, serum or plasma from the subject;
b) heating the first sample to a temperature of 50-85° C.;
c) contacting the first sample, subsequent to the heating of b), with at least one affinity ligand capable of selective interaction with a target protein which immunodetectability is increased by the heating;
d) contacting the second sample, which has not been subjected to the heating, with at least one affinity ligand capable of selective interaction with a target protein which immunodetectability is not increased by the heating;
e) detecting interactions between the antibodies and the corresponding target proteins formed in step c) and d), thereby detecting and/or quantifying proteins in the blood, serum or plasma.

The second aspect is based on the inventors' insight that the immunodetectability of some proteins are improved during the heat treatment of the present disclosure while the immunodetectability of some other proteins is not increased or even decreased. Accordingly, if the detections of the former proteins are performed in a sample that has been heated while the detections of the latter proteins are performed in a non-heated sample, the sensitivity is optimized over a larger range of proteins may be achieved.

The person skilled in the art may without undue burden determine if the immunodetectability of a protein is improved or not by simply performing two measurements of the same protein, one in a heated sample and one in a non-heated sample, and then compare the resulting signals. If the signal is higher in the heat-treated sample, the protein is selected as a protein which immunodetectability is increased by the heating, and the corresponding affinity ligand is contacted with the heat-treated sample in future analyses.

As a third aspect of the present disclosure, there is provided a method for identification of a biomarker of a medical condition, comprising
a) providing blood, serum or plasma samples from a first group of subjects having the medical condition and a second group of subjects not having the medical condition,
b) heating the samples, optionally after dilution, to a temperature of 50-85° C.,
c) contacting the samples, after the heating, with at least one affinity ligand capable of selective interaction with at least protein to determine the levels of the at least one protein in the respective groups,
d) comparing the levels to identify a protein which is occurs to a higher or lower degree in the samples from the first group than in the samples from the second group, thus identifying the biomarker of the medical condition.

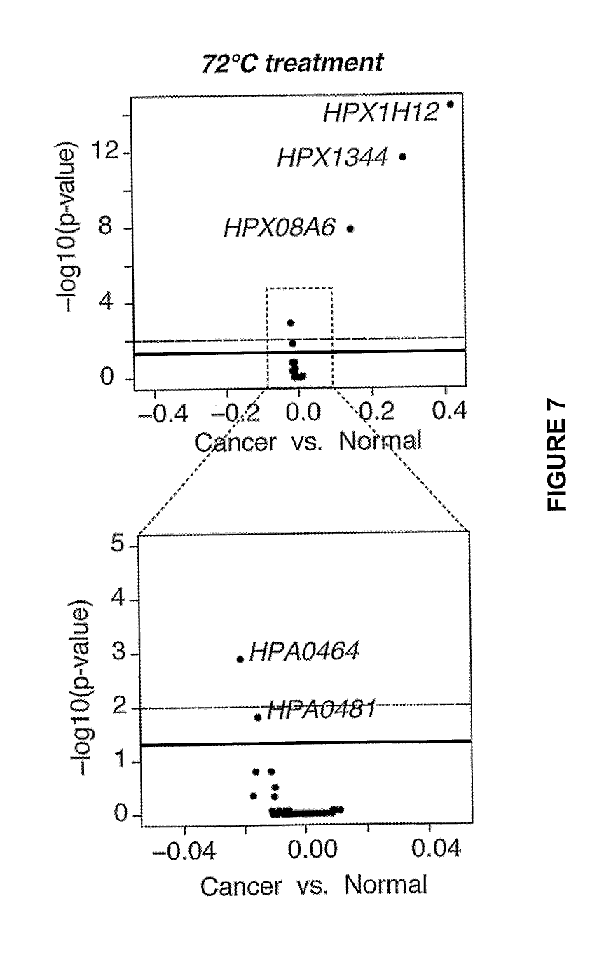

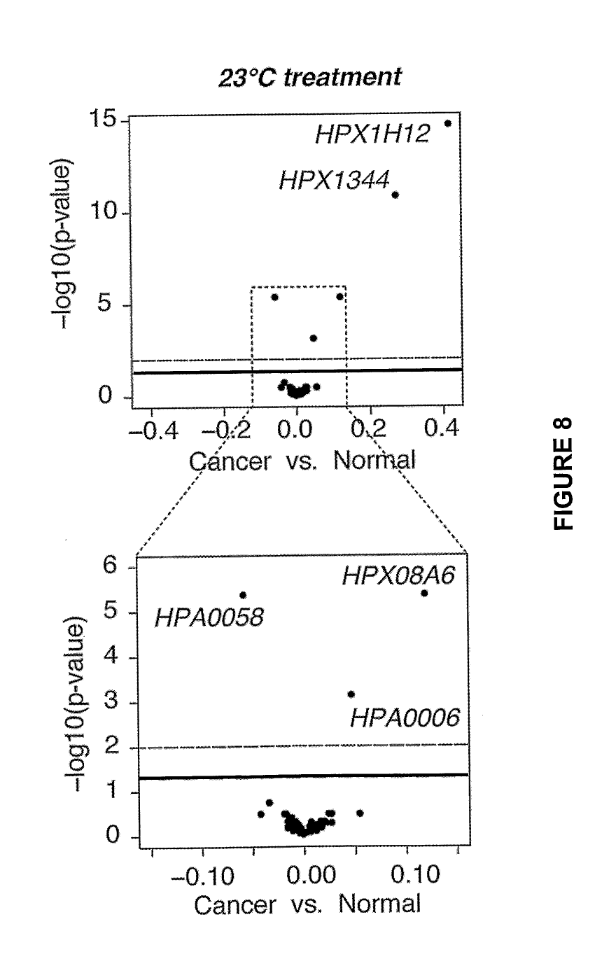

The third aspect of the present disclosure is based on the inventors' finding that a comparison of heated samples from normal and diseased patients revealed differences in protein expression that were not detectable when non-heated samples were analyzed. Consequently, previously unrecognizable biomarkers may be identified using the method of the present disclosure. This is illustrated in FIGS. 7 and 8, in which levels of certain proteins in heated and non-heated samples from normal subjects and subjects having prostate cancer are shown, and two protein biomarkers of prostate cancer are identified.

The medical condition of the third aspect may for example be disease or another medical disorder, such as a cancer.

The person of skill in the art understands how to compare the levels from the two groups in step d) and determine whether a difference between the levels is sufficient for concluding that the protein is a biomarker.

In one embodiment of the third aspect, a protein is identified as a biomarker in step d) if its concentration is at least 25% higher, such as at least 50% higher, such as at least 100% higher, in the samples from the first group than in the samples of the second group. Further, the protein may be identified as a biomarker if the signal from it in a detection system is at least 25% higher, such as at least 50% higher, such as at least 100% higher, in the samples from the first group than in the samples of the second group. Here, the concentration or signal to be compared may be a mean or median value of the concentrations in the samples. Also, the situation may be that the protein is only detected in some of the samples, and in such case, a protein may be identified as a biomarker in step d) if it is detected in a higher percentage of the samples from the first group than from the second group.

A protein may also be identified as a biomarker if it is detected to a lower degree in samples from the diseased group.

In embodiments of the present disclosure, the methods relate only to the detection and/or quantification of non-immunoglobulin proteins of blood, plasma or serum. The detectability of some epitopes of an immunoglobulin protein is affected by whether the immunoglobulin protein is associated with its antigen or not. Without being bound by any specific scientific theory, the inventors believe that the formation or dissociation of such interactions of the target protein is not the source of the effect of the heating. In addition, heating the sample could alter the binding activity of immunoglobulin proteins and thereby compromise the identification of their functionality. Consequently, in embodiments of the methods of the present disclosure, the at least one affinity ligand is capable of selective interaction with at least one non-immunoglobulin protein.

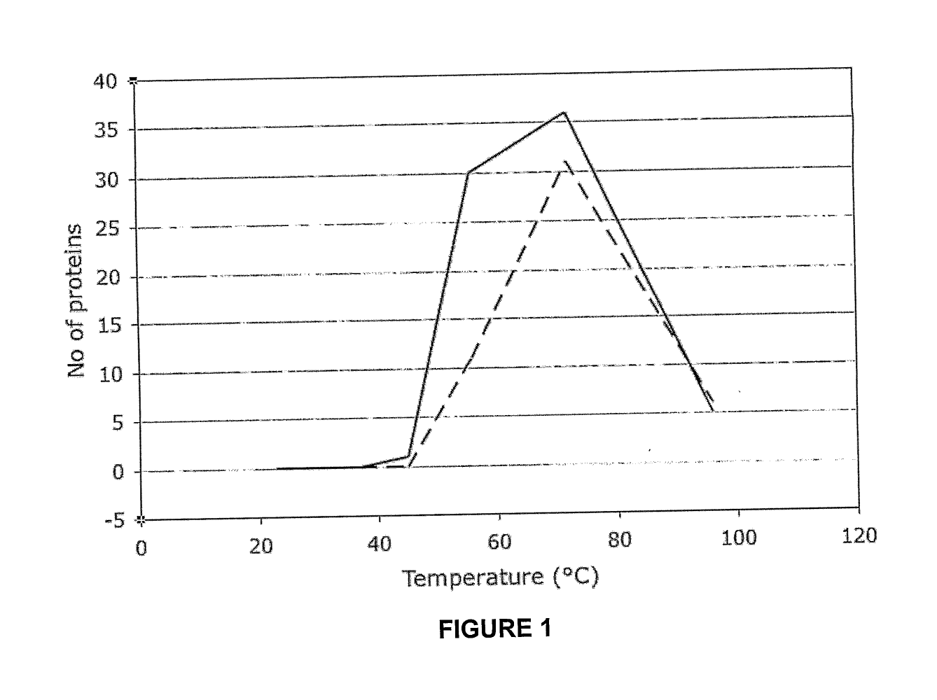

In the methods of the present disclosure, samples are heated to temperature of 50-85° C. In the example embodiments below and FIG. 1 such a temperature range is shown to result in increased immunodetectability. In embodiments of the methods of the present disclosure, the samples are heated to a temperature of 64-85° C., such as 66-78° C., such as 70-74° C., such as about 72° C. In the example embodiments below and FIGS. 1, 3 and 4, temperatures within such ranges are shown to be better than the wider range of 50-85° C., at least in some aspects.

Normally, the heating of the present disclosure is limited in time, which means that the samples are first heated to a temperature, kept at that temperature for a period of time, and then cooled down, normally to a temperature around room temperature, such as 20-25° C. Consequently, the steps following the heating, such as the contact with the affinity ligand, are normally not performed at the increased temperature. The inventors have found that the heating and optionally the cooling may be performed in a thermo cycler. (A thermo cycler is normally used in PCR.) However, other heating means may also be used.

The time of heating may be kept low in order to provide for an efficient use labor and material and thereby an improved economy of the analyses. For example, this may be beneficial in proteomics wherein many proteins may be analyzed in a large group of subjects. The inventors have shown that heating for periods of less than an hour, or even less than half an hour, are sufficient for obtaining a satisfactory result. Thus, in embodiments of the present disclosure, the heating is performed for a period of 0.5-55 minutes, such as 1-40 minutes, such as 1-29 minutes, such as 5-20 minutes, such as about 15 minutes.

In the examples below, the proteins of the sample are labeled prior to the heating. The label is later reacted with a fluorophore and detected in the final detection step. The inventors have shown that such labeling provides for efficient analysis protocol and that labeling prior to the heating results in a higher number of proteins having an increased immunodetectability than labeling after the heating. Further, the sample may be diluted (e.g. 10-100 times) between the labeling and the heating.

In embodiments of the present disclosure, proteins of the sample may thus be labeled prior to the heating with a label that is directly or indirectly detectable in a detection step following the contact between the sample and the at least one affinity ligand. The label may for example comprise biotin, That is, the labeling may for example be biotinylation. The label may be detectable in itself (directly) or through a secondary label (indirectly). Thus, in embodiments of the methods of the present disclosure, the labeled protein may, after the contact with the affinity ligand(s), be contacted with a secondary label which is detectable in a subsequent detection step. The secondary label may for example be a fluorophore.

The sample of the methods of the present disclosure may be diluted before the heating. Dilution is generally considered to decrease the background signal (the noise) as well as the signal from the target protein in analyses based on immunological detection. In embodiments of the present disclosure, the sample may be diluted 10-10000 times, such as 100-2500 times, such as 200-1000 times, such as about 500 times before the heating.

The sample may for example be diluted using a buffer comprising additives, such as rabbit IgG and/or casein. These additives may quench unspecific bindings between proteins in the sample and the (specific) affinity ligand(s), thereby reducing the noise in the detection.

The concentration of the quenching antibody in the buffer may be 0.05-5 mg/ml, such as 0.1-2 mg/ml, such as about 0.5 mg/ml, and the concentration of casein in the buffer may be 0.01-10% (w/v), such as 0.05-2% (w/v), such as about 0.1% (w/v).

In order to provide for concurrent detection of several proteins and thereby an efficient analysis of the protein contents of blood, sample or plasma, the sample may be contacted with more than one affinity ligand in the same reaction compartment. To detect more than 30 different proteins in the same sample, it is beneficial to use affinity ligands in miniaturized and parallelized systems where the ligands could be coupled to beads that are analyzed in a subsequent detection step. Further, such beads may be provided with an identity linking the signal in the subsequent detection step to the affinity ligand and, in turn, the protein. Sometimes such beads are referred to as “coded particles”; see Kingsmore, S. F. Nat Rev. Drug Discovery 2006, 5(4), 310-320. Using traditional sandwich assays, it is in practice very difficult and time consuming to provide a set-up capable of detecting more than 30 different proteins from the same sample.

Consequently, in embodiments of the methods of the present disclosure, the heating is performed prior to a contact between the sample and at least 4, such as at least 10, such as at least 30, such as at least 50 different affinity ligands.

By employing a directly or indirectly detectable label coupled to the proteins and detectable moiety having an identity coupled to the affinity ligand(s), the number of false negatives in the detection step may be kept low since any entity not providing signals from the label associated with the protein and the moiety associated with the affinity ligand may be ignored.

The embodiments presented above are mainly described with reference to the first aspect. However, the person skilled in the art understands that the embodiments also apply to the second and the third aspect mutatis mutandis.

In the context of the present disclosure, “at least one affinity ligand” refers to at least one kind of affinity ligand, wherein the kind is defined by the specificity of the affinity ligand. Thus, the person of skill in the art understands that one kind of affinity ligand may refer to a group of polyclonal antibodies all being capable of selective interaction with the same antigen.

Accordingly, “different affinity ligands” refer to affinity ligands having different specificities.

It is regarded as within the capabilities of those of ordinary skill in the art to select or manufacture the proper affinity ligand and to select the proper format and conditions for detection and/or quantification according to the present disclosure. Nevertheless, examples of affinity ligands that may prove useful, as well as examples of formats and conditions for detection and/or quantification, are given below for the sake of illustration.

Thus, in embodiments of the present disclosure, the affinity ligand may be selected from the group consisting of antibodies, fragments thereof and derivatives thereof, i.e., affinity ligands based on an immunoglobulin scaffold. The antibodies and the fragments or derivatives thereof may be isolated and/or mono-specific. Antibodies comprise monoclonal and polyclonal antibodies of any origin, including murine, rabbit, human and other antibodies, as well as chimeric antibodies comprising sequences from different species, such as partly humanized antibodies, e.g., partly humanized mouse antibodies. Polyclonal antibodies may be produced by immunization of animals with the antigen of choice. Monoclonal antibodies of defined specificity can be produced using the hybridoma technology developed by Köhler and Milstein (Köhler G and Milstein C (1976) Eur. J. Immunol. 6:511-519). The antibody fragments and derivatives of the present disclosure are capable of selective interaction with the same antigen as the antibody they are fragments or derivatives of. Antibody fragments and derivatives comprise Fab fragments, consisting of the first constant domain of the heavy chain (CH1), the constant domain of the light chain (CL), the variable domain of the heavy chain (VH) and the variable domain of the light chain (VL) of an intact immunoglobulin protein; Fv fragments, consisting of the two variable antibody domains VH and VL (Skerra A and Plückthun A (1988) Science 240:1038-1041); single chain Fv fragments (scFv), consisting of the two VH and VL domains linked together by a flexible peptide linker (Bird R E and Walker B W (1991) Trends Biotechnol. 9:132-137); Bence Jones dimers (Stevens F J et al. (1991) Biochemistry 30:6803-6805); camelid heavy-chain dimers (Hamers-Casterman C et al. (1993) Nature 363:446-448) and single variable domains (Cai X and Garen A (1996) Proc. Natl. Acad. Sci. U.S.A. 93:6280-6285; Masat L et al. (1994) Proc. Natl. Acad. Sci. U.S.A. 91:893-896), and single domain scaffolds like e.g., the New Antigen Receptor (NAR) from the nurse shark (Dooley H et al. (2003) Mol. Immunol. 40:25-33) and minibodies based on a variable heavy domain (Skerra A and Plückthun A (1988) Science 240:1038-1041).

In the context of the present disclosure, a “mono-specific antibody” is one of a population of polyclonal antibodies which has been affinity purified on its own antigen, thereby separating such mono-specific antibodies from other antiserum proteins and non-specific antibodies. This affinity purification results in antibodies that bind selectively to its antigen. To obtain mono-specific antibodies that can be used in the method of the present disclosure, polyclonal antisera may be purified by a two-step immunoaffinity based protocol to obtain mono-specific antibodies selective for the target protein. Antibodies directed against generic affinity tags of antigen fragments are removed in a primary depletion step, using the immobilized tag protein as the capturing agent. Following the first depletion step, the serum is loaded on a second affinity column with the antigen as capturing agent, in order to enrich for antibodies specific for the antigen (see also Nilsson P et al. (2005) Proteomics 5:4327-4337).

Polyclonal and monoclonal antibodies, as well as their fragments and derivatives, represent the traditional choice of affinity ligands in applications requiring selective biomolecular recognition, such as in the detection and/or quantification of protein according to the present disclosure. However, those of skill in the art know that, due to the increasing demand of high throughput generation of selective binding ligands and low cost production systems, new biomolecular diversity technologies have been developed. This has enabled a generation of novel types of affinity ligands of both immunoglobulin as well as non-immunoglobulin origin that sometimes have proven to be equally useful as binding ligands in biomolecular recognition applications and can be used instead of, or together with, immunoglobulins.

The biomolecular diversity needed for selection of affinity ligands may be generated by combinatorial engineering of one of a plurality of possible scaffold molecules, and specific and/or selective affinity ligands are then selected using a suitable selection platform. The scaffold molecule may be of immunoglobulin protein origin (Bradbury A R and Marks J D (2004) J. Immunol. Meths. 290:29-49), of non-immunoglobulin protein origin (Nygren P A and Skerra A (2004) J. Immunol. Meths. 290:3-28), or of an oligonucleotide origin (Gold L et al. (1995) Annu. Rev. Biochem. 64:763-797).

A large number of non-immunoglobulin protein scaffolds have been used as supporting structures in development of novel binding proteins. Non-limiting examples of such structures, useful for generating affinity ligands for use according to the present disclosure, are staphylococcal protein A and domains thereof and derivatives of these domains, such as protein Z (Nord K et al. (1997) Nat. Biotechnol. 15:772-777); lipocalins (Beste G et al. (1999) Proc. Natl. Acad. Sci. U.S.A. 96:1898-1903); ankyrin repeat domains (Binz H K et al. (2003) J. Mol. Biol. 332:489-503); cellulose binding domains (CBD) (Smith G P et al. (1998) J. Mol. Biol. 277:317-332; Lehtiö J et al. (2000) Proteins 41:316-322); γ crystallines (Fiedler U and Rudolph R, WO01/04144); green fluorescent protein (GFP) (Peelle B et al. (2001) Chem. Biol. 8:521-534); human cytotoxic T lymphocyte-associated antigen 4 (CTLA-4) (Hufton S E et al. (2000) FEBS Lett. 475:225-231; Irving R A et al. (2001) J. Immunol. Meth. 248:31-45); protease inhibitors, such as Knottin proteins (Wentzel A et al. (2001) J. Bacteriol. 183:7273-7284; Baggio R et al. (2002) J. Mol. Recognit. 15:126-134) and Kunitz domains (Roberts B L et al. (1992) Gene 121:9-15; Dennis M S and Lazarus R A (1994) J. Biol. Chem. 269:22137-22144); PDZ domains (Schneider S et al. (1999) Nat. Biotechnol. 17:170-175); peptide aptamers, such as thioredoxin (Lu Z et al. (1995) Biotechnology 13:366-372; Klevenz B et al. (2002) Cell. Mol. Life Sci. 59:1993-1998); staphylococcal nuclease (Norman T C et al. (1999) Science 285:591-595); tendamistats (McConell S J and Hoess R H (1995) J. Mol. Biol. 250:460-479; Li R et al. (2003) Protein Eng. 16:65-72); trinectins based on the fibronectin type III domain (Koide A et al. (1998) J. Mol. Biol. 284:1141-1151; Xu L et al. (2002) Chem. Biol. 9:933-942); and zinc fingers (Bianchi E et al. (1995) J. Mol. Biol. 247:154-160; Klug A (1999) J. Mol. Biol. 293:215-218; Segal D J et al. (2003) Biochemistry 42:2137-2148).

The above-mentioned examples of non-immunoglobulin protein scaffolds include scaffold proteins presenting a single randomized loop used for the generation of novel binding specificities, protein scaffolds with a rigid secondary structure where side chains protruding from the protein surface are randomized for the generation of novel binding specificities, and scaffolds exhibiting a non-contiguous hyper-variable loop region used for the generation of novel binding specificities.

In addition to non-immunoglobulin proteins, oligonucleotides may also be used as affinity ligands. Single stranded nucleic acids, called aptamers or decoys, fold into well-defined three-dimensional structures and bind to their target with high affinity and specificity. (Ellington A D and Szostak J W (1990) Nature 346:818-822; Brody E N and Gold L (2000) J. Biotechnol. 74:5-13; Mayer G and Jenne A (2004) BioDrugs 18:351-359). The oligonucleotide ligands can be either RNA or DNA and can bind to a wide range of target molecule classes.

For selection of the desired affinity ligand from a pool of variants of any of the scaffold structures mentioned above, a number of selection platforms are available for the isolation of a specific novel ligand against a target protein of choice. Selection platforms include, but are not limited to, phage display (Smith G P (1985) Science 228:1315-1317), ribosome display (Hanes J and Plückthun A (1997) Proc. Natl. Acad. Sci. U.S.A. 94:4937-4942), yeast two-hybrid system (Fields S and Song O (1989) Nature 340:245-246), yeast display (Gai S A and Wittrup K D (2007) Curr Opin Struct Biol 17:467-473), mRNA display (Roberts R W and Szostak J W (1997) Proc. Natl. Acad. Sci. U.S.A. 94:12297-12302), bacterial display (Daugherty P S (2007) Curr Opin Struct Biol 17:474-480, Kronqvist N et al. (2008) Protein Eng Des Sel 1-9, Harvey B R et al. (2004) PNAS 101(25):913-9198), microbead display (Nord O et al. (2003) J Biotechnol 106:1-13, WO01/05808), SELEX (System Evolution of Ligands by Exponential Enrichment) (Tuerk C and Gold L (1990) Science 249:505-510) and protein fragment complementation assays (PCA) (Remy I and Michnick S W (1999) Proc. Natl. Acad. Sci. U.S.A. 96:5394-5399).

Thus, in embodiments of the present disclosure, the affinity ligand may be a non-immunoglobulin affinity ligand derived from any of the protein scaffolds listed above, or an oligonucleotide molecule.

The detection and/or quantification of the present disclosure may be accomplished in any way known to the skilled person for detection and/or quantification of binding reagents in assays based on interactions between affinity ligands, such as antibodies, and antigen. Accordingly, any affinity ligand described above may be used to quantitatively and/or qualitatively detect the presence of a protein in blood or blood-derived samples. These “primary” affinity ligands may be labeled themselves with various markers or may in turn be detected by secondary, labeled affinity ligands to allow detection, visualization and/or quantification. This can be accomplished using any one or more of a multitude of labels, which can be conjugated to the primary or secondary affinity ligand, using any one or more of a multitude of techniques known to the skilled person, and not as such involving any undue experimentation.

Non-limiting examples of labels that can be conjugated to primary and/or secondary affinity ligands include fluorescent dyes or metals (e.g., fluorescein, rhodamine, phycoerythrin, fluorescamine), chromophoric dyes (e.g., rhodopsin), chemiluminescent compounds (e.g., luminal, imidazole) and bioluminescent proteins (e.g., luciferin, luciferase), haptens (e.g., biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g., horseradish peroxidase, alkaline phosphatase, beta-lactamase), radioisotopes (e.g., ³H, ¹⁴C, ³²P, ³⁵S or ¹²⁵I) and particles (e.g., gold). In the context of the present disclosure, “particles” refer to particles, such as metal particles, suitable for labeling of molecules. Further, the affinity ligands may also be labeled with fluorescent semiconductor nanocrystals (quantum dots). Quantum dots have superior quantum yield and are more photostable compared to organic fluorophores and are therefore more easily detected (Chan et al. (2002) Curr Opi Biotech. 13: 40-46). The different types of labels can be conjugated to an affinity ligand using various chemistries, e.g., the amine reaction or the thiol reaction. However, other reactive groups than amines and thiols can be used, e.g., aldehydes, carboxylic acids and glutamine.

The method aspects above may be put to use in any of several known formats and set-ups, of which a non-limiting selection is discussed below.

The method of visualization of labels on the affinity ligand may include, but is not restricted to, fluorometric, luminometric and/or enzymatic techniques. Fluorescence is detected and/or quantified by exposing fluorescent labels to light of a specific wavelength and thereafter detecting and/or quantifying the emitted light in a specific wavelength region. The presence of a luminescently tagged affinity ligand may be detected and/or quantified by luminescence developed during a chemical reaction. Detection of an enzymatic reaction is due to a color shift in the sample arising from chemical reaction. Different types of ELISA:s are examples of methods based on an enzymatic reaction. Those of skill in the art are aware that a variety of different protocols can be modified in order for proper detection and/or quantification.

Without being bound by any specific scientific theory, the inventors believe that the immunodetectability of linear epitopes is increased using the heating of the present disclosure. In embodiments of the methods of the present disclosure, the affinity ligands may thus be capable of selective interaction with linear/continuous epitopes. As an example, an antibody capable of selective interaction with a linear/continuous epitope may be produced by immunizing an animal with a peptide that comprises the epitope but is not forming a (rigid) secondary structure. The antibodies employed in the examples section below were produced using Protein Epitope Signature Tags (PrESTs) immunizations, which frequently results in antibodies recognizing linear/continuous epitopes.

### DETAILED DESCRIPTION OF EXAMPLE EMBODIMENTS

### Materials and Methods

### Bead Coupling

Mono-specific antibodies were coupled to carboxylated beads (COOH Micorspheres, Luminex-Corp.) in accordance to the manufacturer's protocol with minor modifications. For the prostate cancer approach each of the antibodies, 3.2 μg were coupled to 10⁶ beads using centrifugal filter units (Ultrafree-MC, Millipore) to a final concentration of 40 μg/ml. Beads were stored in a protein containing buffer (Blocking Reagent for ELISA, Roche) with NaN₃. All coupled beads were re-suspended with sonication in an ultrasonic cleaner (Branson, Ultrasonic Corporation) for 5 min prior to storage at 4° C. A 100-plex bead mixture was created in solution, optimized as previously described (Schwenk et al (2007) Mol Cell Proteomics 6, 125-132) and utilized through-out the studies.

For the first and second approach presented below, magnetic carboxylated beads (MagPlex Micorspheres, Luminex-Corp.) were utilized. The difference to the protocol stated above is that the beads were coupled in a microtiter plate (Greiner Bioone) and that beads were washed placing the plate onto a magnet (LifeSept, Dexter). Finally a 76-plex bead mixture was created in solution.

### Serum and Plasma Labeling and Assay Procedure.

At first, samples (plasma or serum) were thawed at RT and centrifuged for 10 min at 10,000 rpm. 30 μl of each sample was transferred into a microtiter plate (Abgene), then plates were sealed, vortexed and centrifuged (1 min at 3,000 rpm). Next, 3 μl of each sample was transferred into a new microtiter plate, followed by addition of 22 μl 1× PBS to each sample using a liquid handler (Plate mate 2×2, Matrix), then plates were sealed, vortexed and centrifuged (1 min at 3,000 rpm). Subsequently, N-hydroxysuccinimidyl ester of biotinoyl-tetraoxapentadecanoic acid (NHS-PEO4-Biotin, Pierce) was added at 10-fold molar excess to yield an overall 1/10 sample dilution followed by an incubation over 2 h at 4° C. in a microtiter plate shaker (Thermomixer, Eppendorf). The reaction was stopped by the addition of a 250-fold molar excess of Tris-HCl, pH 8.0 over biotin and incubated for another 20 min at 4° C. Samples were then used immediately or stored at −80° C.

All samples were utilized without removing unincorporated biotin and diluted 1/50, i.e. 1 μl sample and 49 μl assay buffer composed of 0.5% (w/v) polyvinylalcohol and 0.8% (w/v) polyvinylpyrrolidone (Sigma) in 0.1% (w/v) casein in PBS (PVXC) supplemented with 0.5 mg/ml non-specific rabbit IgG (Bethyl). As controls, a nonspecific rabbit IgG (Jackson ImmunoResearch) and a HSA binding Affibody (Affibody AB) were included Next the plates were heat-treated at 72° C. for 15 min followed by a 15 min incubation at 23° C. in a thermo cycler (DNA Engine Tetrad cycler, PTC225, BioRad). The plates were then centrifuged (1 min at 3,000 rpm) and 45 μl of each sample was added to 5 μl of bead mixtures. Incubation took place over night on a shaker at 23° C. and this was followed by the beads being washed in wells with 3×50 μl PBST (1× PBS pH 7.4, 0.1% Tween20)

For first and second approach pursued with magnetic beads, a microtiter plate (Greiner Bioone) was use in connection to magnetic bead sedimentation for plate washing. For the stated prostate cancer approach, a filter bottomed microtiter plate (Millipore) was employed and a vacuum device (Millipore) was utilized for washing the beads.

Washing was followed by a 10 min incubation with 50 μl of a stop solution containing 0.1% paraformaldehyde in PBS. Next, 1×50 μl PBST and 50 μl of 0.5 μg/ml R-Phycoerythrin labeled streptavidin (Invitrogen) in PBST were added to the bead-mix and incubated for 20 min on a shaker at 23° C. Finally, wells were washed in 3×50 μl PBST and measured in 100 μl PBST.

### Readout and Data Analysis

Measurements were performed on Luminex LX200 instrumentation using Luminex IS 2.3 software counting 100 events per color code ID for each single specificity analysis. The coupling efficiency for each antibody was determined via R-Phycoerythrin labeled anti-rabbit IgG antibody (Jackson ImmunoResearch). To display antibody-protein interactions the median fluorescence intensity (MFI) was chosen. Data analyses and graphical representations were performed using Microsoft Office Excel 2003 or R, a language and environment for statistical computing and graphics (Ihaka, R et al (1996) J. Comput. Graph. Stat. 5, 299-3214).

### Results

### a) First Approach

Serum and plasma samples from one normal patient were obtained through the EU project MolPAGE.

The studied antibodies were selected targeting different classes of proteins such as known serum proteins. In total, 135 monospecific antibodies (msAbs) were included in this study targeting the products of 93 unique protein encoding genes. The mono-specific antibodies were obtained from the HPA project (www.proteinatlas.org). As controls, a nonspecific rabbit IgG (Jackson ImmunoResearch) and a HSA binding Affibody (Affibody AB) were included. All serum and plasma samples were analyzed in triplicates. Herewith, one time interval per temperature was investigated for both plasma and serum. The samples were dilutes, labeled and prepare in assay buffer accordingly. Then the treatments included 30 min at 23° C., 37° C., 45° C., 56° C., as well as 15 min at 72° C. and 5 min at 96° C. All samples were cooled to 23° C. after the respective heat treatment intervals and then combined with the bead mixtures.

For optimization of immunodetectability, the samples were heat-treated at a range of temperatures subsequent to biotinylation. Temperatures included in the test were: 23° C., 37° C., 45° C., 56° C., 72° C. and 96° C. In FIG. 1, the number of proteins exhibiting an at least two-fold signal intensity increase is plotted against heat-treatment temperatures. 72° C. resulted in the highest number of proteins with the increased signal. 56° C. also resulted in a considerable number of proteins with increased signal, but the result was not superior to 72° C., especially in the serum sample. 96° C. only resulted in a few proteins with increased signal.

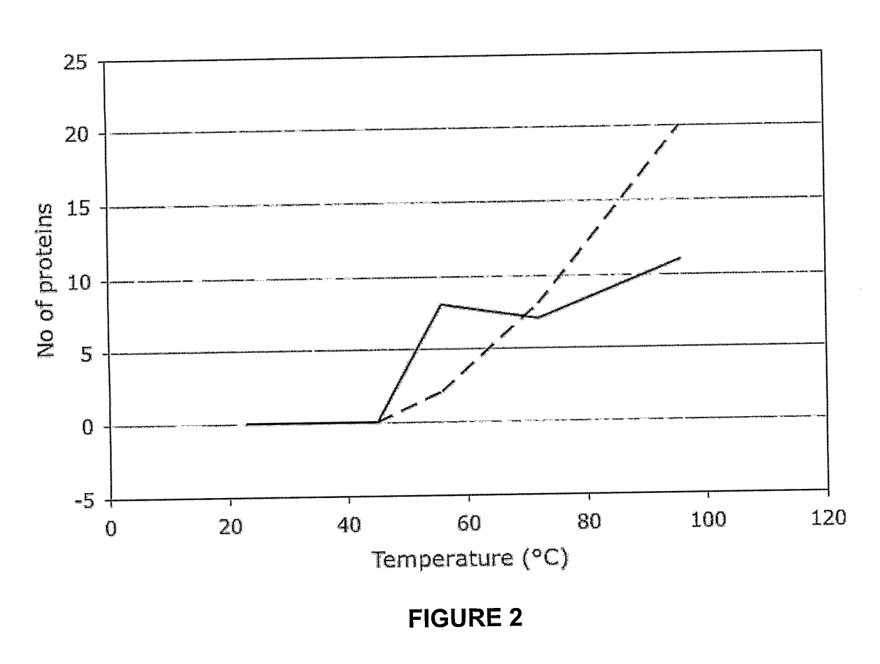

In FIG. 2, the negative effect of the heating is shown by a plot of the number of proteins exhibiting an at least two-fold signal intensity decrease against heat treatment temperatures. Here, 96° C. is shown to result in a relatively high number of proteins with decreased signal, especially in the serum sample. At 96° C., the number of proteins with decreased signal was in fact higher than the number of protein with increased signal in both the serum sample and the plasma sample. Raising the temperature from 56° C. to 72° C., the number of proteins with decreased signal was increased in serum, but decreased in plasma.

Heating to 37° C. and 45° C. showed to be of no or only minor impact on the signal intensities.

### b) Second Approach

Serum and plasma samples from one normal patient were obtained through the EU project MolPAGE.

The studied antibodies were selected targeting different classes of proteins such as known serum proteins. In total, 135 monospecific antibodies (msAbs) were included in this study targeting the products of 93 unique protein encoding genes. The mono-specific antibodies were obtained from the HPA project (www.proteinatlas.org). As controls, a nonspecific rabbit IgG (Jackson ImmunoResearch) and a HSA binding Affibody (Affibody AB) were included. Herewith, four time intervals for two temperatures were investigated for both plasma and serum. The samples were dilutes, labeled and prepare in assay buffer accordingly. Then the intervals of 5, 10, 15 and 30 min were chosen for both 56° C. and 72° C. to be compared to 30 min at 23° C. All samples were cooled to 23° C. after the respective heat treatment intervals and then combined with the bead mixtures. For treatments 30 min at 23° C., 30 min at 56° C. and 15 min at 72° C. samples were analyzed as triplicates,

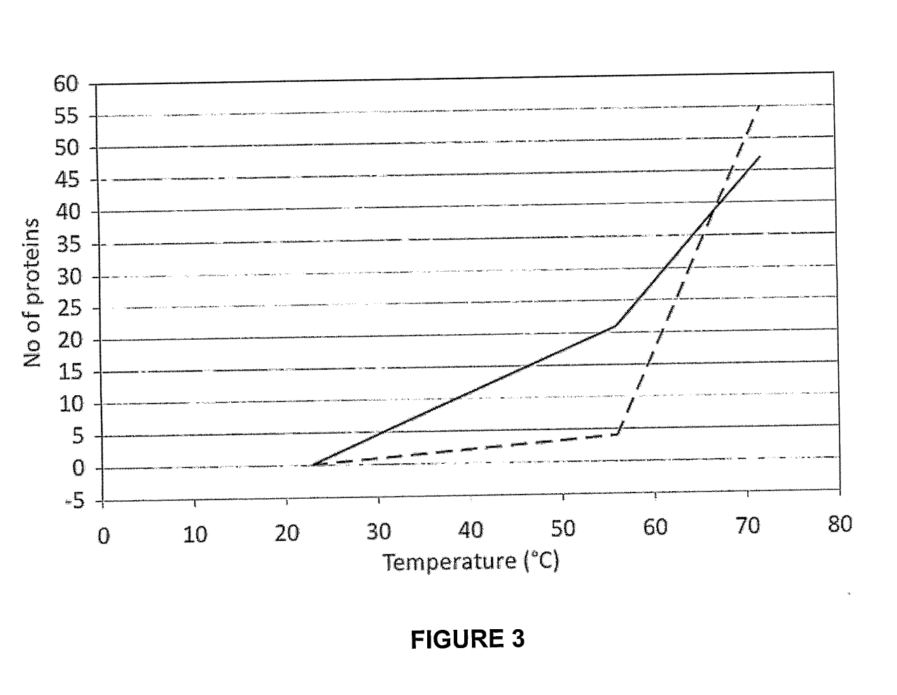

In FIG. 3, the number of proteins exhibiting an at least two-fold signal intensity increase is plotted against heat treatment temperatures like in the first approach. Again, 72° C. resulted in the highest number of proteins with the increased signal in both serum and plasma. 56° C. resulted in a considerable number of proteins with increased signal in plasma, but the result was not as good as for 72° C.

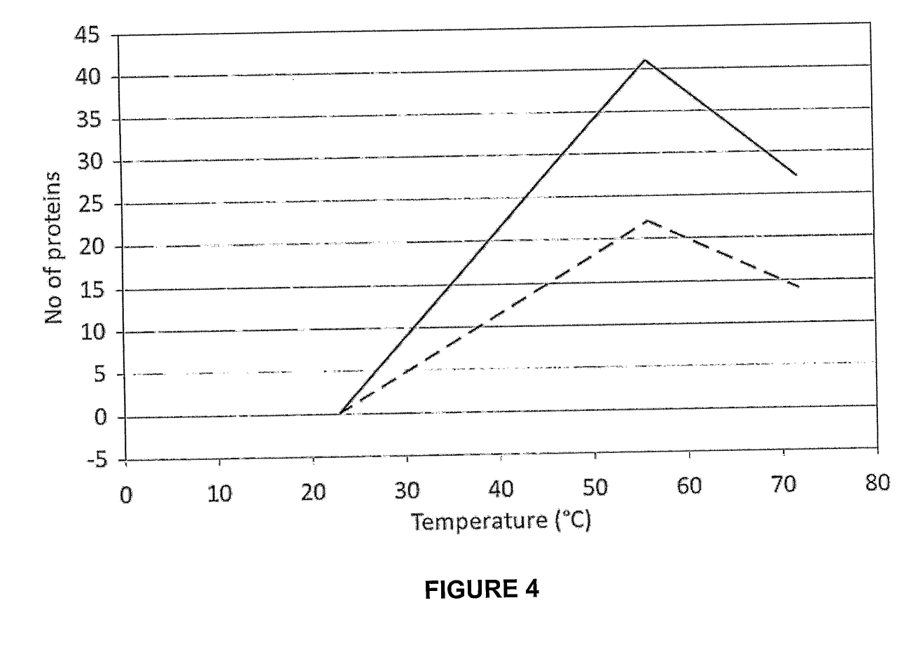

In FIG. 4, the negative effect of the heating of the second approach is shown by a plot of the number of proteins exhibiting an at least two-fold signal intensity decrease against heat treatment temperatures. Raising the temperature from 56° C. to 72° C., the number of proteins with decreased signal was smaller for both serum and plasma. This was surprising since proteins are generally believed to be more susceptible to precipitation alongside heat induced coagulation.

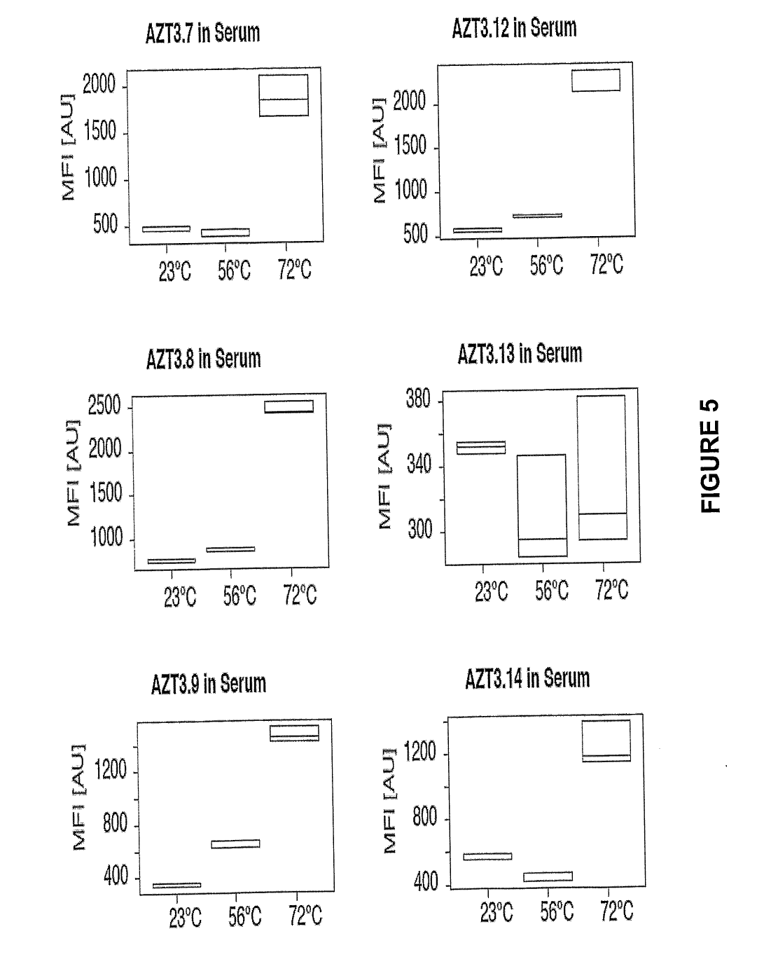

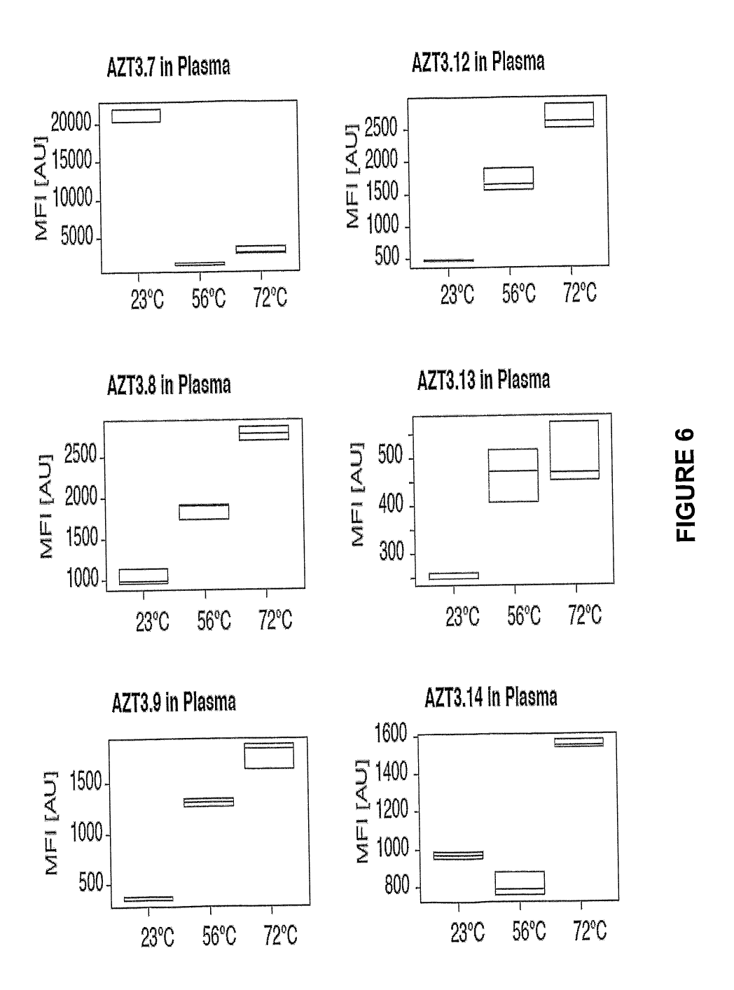

FIG. 5 shows the detection levels of six of the 92 proteins targeted in serum after heat-treatment at 23° C., 56° C. and 72° C. FIG. 6 shows the corresponding results achieved in plasma. The data was summarized in box plots using the non-normalized median fluorescent intensity levels. The signal intensities from individual target protein are directly compared after treatment at 23° C. for 30 min, heating to 56° C. for 30 min and heating to 72° C. for 15 min, respectively. The signal intensities of some of the proteins decreased after heating while the signal intensities some other proteins increased. However, it is noteworthy that signals are higher after the 72° C.-treatment than after the 56° C.-treatment for all proteins of the figure. In fact, out of all the proteins detected in the second approach, the signal was only decreased when raising the temperature from 56° C. to 72° C. for one single protein.

In conclusion, heating to a temperature in the range of from about 50° C. to about 85° C. appears to have a beneficial effect on protein analysis in blood or blood-derived samples when using immunological detection. A temperature range around 72° C., such as 64-85° C., 66-78° C. or 70-74° C. appears to be particularly beneficial.

### c) Prostate Cancer Cohort

Plasma samples were obtained from the pathology departments at Lund and Malmö University Hospitals, Sweden. From male patients undergoing a routine test for levels of prostate specific antigen (PSA) 20 samples with normal PSA levels and 20 samples with high PSA levels were collected. The latter group had PSA levels between 60-3,000 ng/ml which serves as an indicator for prostate cancer, so this group was referred to as the cancer group. The group of normals had PSA levels <1.5 ng/ml. Beyond the stated PSA levels no patient information was accessible and anonymized samples had been obtained to agree with ethical requirements.

The studied antibodies were selected without any previous disease preference and only due to their performance in validation procedures used within the HPA project (www.proteinatlas.org). In total, 96 monospecific antibodies (msAbs) were included in this study targeting 95 different serum proteins. In addition, three anti-PSA antibodies (HPX antibodies) were obtained from Roche (Basel) and HyTest (Finland) and included as positive controls. All plasma samples were analyzed in a randomized layout and the obtained intensity values were processed via log2-transformation, integral normalization, and probabilistic quotient normalization.

Proteins exhibiting significantly different detection between normal- and cancer samples were identified by Student's t-test and in connection to relative fold changes to be visualized by volcano plots, as shown in FIGS. 7 and 8. The plot displays normalized fluorescence intensity ratios (x-axis) and the corresponding false discovery rate corrected P-values (y-axis), reflecting the significance of how certain proteins are differentially detected. The lower the P-value for each target, the higher the probability of that the protein is differentially present. The horizontal lines inside the plot indicate commonly used P-values of 0.05 and 0.01, respectively.

In FIG. 7 samples were heat-treated at 72° C. as described in the material and method section. The three HPX antibodies were found to be detecting PSA at a significantly higher level in the cancer group (high PSA level) as compared to normal patients (low PSA level). In addition, two new marker proteins, HPA0464 (p<0.01) and HPA0481 (p<0.05), appeared to be down regulated in the cancer group and were only found post 72° C. heating. In FIG. 8, samples where not heat-treated, and for these patients, the overall significance for anti-PSA antibodies was reduced. Here, two new proteins were discovered among which target levels of HPA0006 were higher and of HPA0058 were lower in patients with an elevated risk of being diagnosed with prostate cancer. Consequently, the immunodetectability of some proteins was increased by the heating while the immunodetectability of some other proteins was better after no heating.

In conclusion, two proteins (HPA0464 and HPA0481) were detected at a lower degree in heat-treated samples from subjects having prostate cancer than in heat-treated samples from the healthy subjects (FIG. 7). However, this difference is not seen in non-heat-treated samples (FIG. 8). Consequently, heat-treatment appears to be necessary for identifying the two proteins as biomarkers for prostate cancer in this type of analysis.

1. Method for improving the immunodetectability of at least one protein in an optionally diluted sample of blood, serum or plasma, comprising a step of heating the sample to a temperature of 50-85° C. prior to a contact between the sample and at least one affinity ligand for detection and/or quantification of the at least one protein, wherein proteins of the sample are labeled prior to the heating with a label that is directly or indirectly detectable in a direction step following the contact between the sample and the at least one affinity ligand.

2. Method according to claim 1, wherein the sample is heated to a temperature of 66-78° C.

3. Method according to claim 1, wherein the heating is performed prior to a contact between the sample and at least 4 different affinity ligands.

4. Method according to claim 1, wherein the temperature is maintained for 0.5-45 minutes.

5. (canceled)

6. Method according to claim 1, wherein the label contains biotin.

7. Method according to claim 1, wherein the sample is diluted 10-10000 times before the heating.

8. Method according to claim 7, wherein the sample is diluted with a buffer comprising rabbit IgG and/or casein.

9. Method according to claim 8, wherein the concentration of rabbit IgG in the buffer is 0.05-5 mg/ml and the concentration of casein in the buffer is 0.01-1% (w/v).

10. Method according to claim 1, wherein the at least one affinity ligand is at least one antibody.

11. Method according to claim 1, wherein the at least one affinity ligand is coupled to an identifiable moiety, such as an identifiable bead.

12. Method for detecting and/or quantifying proteins in blood, serum or plasma from a subject, comprising:
a) providing a first and a second optionally diluted sample of blood, serum or plasma from the subject;
b) heating the first sample to a temperature of 50-85° C.;
c) contacting the first sample, subsequent to the heating of b), with at least one affinity ligand capable of selective interaction with a target protein which immunodetectability is increased by the heating;
d) contacting the second sample, which has not been subjected to the heating, with at least one affinity ligand capable of selective interaction with a target protein which immunodetectability is not increased by the heating;
e) detecting interactions between the antibodies and the corresponding target proteins formed in step c) and d), thereby detecting and/or quantifying proteins in the blood, serum or plasma.

13. Method for identification of a biomarker of a medical condition, comprising
a) providing blood, serum or plasma samples from a first group of subjects having the medical condition and a second group of subjects not having the medical condition,
b) heating the samples, optionally after dilution, to a temperature of 66-78° C.
e) contacting the samples after the heating with at least one affinity ligand capable of selective interaction with at least one protein to determine the levels of the at least one protein in the respective groups,
d) comparing the levels to identify a protein which occurs to a higher or lower degree in the sample from the first group than in the samples from the second group, thus identifying the biomarker of the medical condition.

14. Method according to claim 1, wherein the sample is heated to a temperature of 64-85° C.

15. Method according to claim 1, wherein the sample is heated to a temperature of 70-74° C.

16. Method according to claim 1, wherein the heating is performed prior to a contact between the sample and at least 30 different affinity ligands.

17. Method according to claim 1, wherein the temperature is maintained for 1-29 minutes.

18. Method according to claim 1, wherein the temperature is maintained for 5-20 minutes.

19. Method according to claim 1, wherein the sample is diluted 100-2500 times before the heating.

20. Method according to claim 12, wherein the at least one affinity ligand of step c) is at least one antibody and the at least one affinity ligand of step d) is at least one antibody.

21. Method according to claim 13, wherein the at least one affinity ligand is at least one antibody.
